(19) United States
(12) Patent Application Publication
(10) Pub. No.: US 2025/0263493 A1
(43) Pub. Date: Aug. 21, 2025

(54) ANTI-CD40L ANTIBODIES AND METHODS FOR TREATING CD40L-RELATED DISEASES OR DISORDERS

(71) Applicant: ALS Therapy Development Institute, Watertown, MA (US)

(72) Inventors: John M. Lincecum, Jamaica Plain, MA (US); Bingbing Jiang, Brighton, MA (US); Steven N. Perrin, Newbury, MA (US); Alan Gill, Reading, MA (US); Cynthia A. Gill, Reading, MA (US); Fernando G. Vieira, Boston, MA (US)

(21) Appl. No.: 19/030,443

(22) Filed: Jan. 17, 2025

Related U.S. Application Data
(63) Continuation of application No. 18/341,202, filed on Jun. 26, 2023, now Pat. No. 12,275,793.
Application No. 18/341,202 is a division of application No. 17/322,486, filed on May 17, 2021, now Pat. No. 11,692,040.
Application No. 17/322,486 is a division of application No. 15/931,315, filed on May 13, 2020, now Pat. No. 11,014,990.
Application No. 15/931,315 is a division of application No. 16/125,317, filed on Sep. 7, 2018, now Pat. No. 10,683,356.
Application No. 16/125,317 is a division of application No. 15/667,477, filed on Aug. 2, 2017, now Pat. No. 10,106,618.
Application No. 15/667,477 is a continuation of application No. PCT/US2016/016165, filed on Feb. 2, 2016.
(60) Provisional application No. 62/111,261, filed on Feb. 3, 2015.

(51) Int. Cl. C07K 16/28; A61K 39/395; A61K 45/06

(52) U.S. Cl. CPC C07K 16/2875; A61K 39/3955; A61K 45/06; C07K 2317/24; C07K 2317/52; C07K 2317/71; C07K 2317/76; C07K 2317/94

(57) ABSTRACT

Anti-human CD40L antibodies engineered to lack the ability to activate platelets and methods for treating patients having a CD40L-associated disease.

### CROSS REFERENCE TO RELATED APPLICATIONS

This application is a divisional application of U.S. patent application Ser. No. 17/322,486, filed on May 17, 2021, which is a divisional application of U.S. patent application Ser. No. 15/931,315, filed May 13, 2020, which issued as U.S. Pat. No. 11,014,990 on May 25, 2021, which is a divisional application of U.S. patent application Ser. No. 16/125,317, filed on Sep. 7, 2018, which issued as U.S. Pat. No. 10,683,356 on Jun. 16, 2020, which is a divisional application of U.S. patent application Ser. No. 15/667,477, filed on Aug. 2, 2017, which issued as U.S. Pat. No. 10,106,618 on Oct. 23, 2018, which is a continuation application of, and claims priority to, PCT International Application No. PCT/US2016/016165, filed Feb. 2, 2016, which claims the benefit under 35 USC § 119 of U.S. Provisional Patent Application No. 62/111,261, filed Feb. 3, 2015, the entire contents of the aforementioned disclosures are hereby incorporated by reference.

### FIELD

Anti-CD40L antibodies, compositions comprising the antibodies, and method of using same for treatment of CD40L-related diseases ordisorders.

### SEQUENCE LISTING

This application contains a Sequence Listing which is submitted herewith in electronically readable format. The electronic Sequence Listing file was created on Jun. 15, 2023 is named “ELDN.007D4.xml” and has a size of 39,961 bytes. The entire content of the Sequence Listing in the electronic “ELDN.007D4.xml” file is incorporated herein by this reference.

### BACKGROUND

The interaction of CD40 with its ligand CD40L plays a critical role in regulating immune responses. Binding of CD40L to CD40 triggers activation of the CD40 pathway which up-regulates costimulatory molecules such as CD80 and CD86. Blockade of the interaction between CD40 and CD40L by monoclonal antibodies has been shown to result in protection from autoimmunity and graft rejection in various preclinical models. Recently, in a mouse model of amyotrophic lateral sclerosis, an antibody directed to CD40L was shown to delay disease onset and prolong survival the onset of disease (U.S. Pat. No. 8,435,514, hereby incorporated by reference). In early clinical studies, the humanized anti-CD40L antibody hu5c8 showed efficacy in patients with lupus and in patients with immune thrombocytopenic purpura. However, incidents of thromboembolism in the patients treated with hu5c8 halted further trials. Further in vitro and preclinical animal studies established that interaction of the Fc with the Fc receptor Fc-gamma RIIa caused platelet activation, and aggregation, that resulted in thromboembolic events. Various approaches have been taken to reduce or eliminate the interaction of the immunoglobulin Fe region with Fc-gamma RIIa, including introducing a point mutation in the Fc region to make an alpha-glycosylated anti-IC40L IgG1 which lacked Fc effector function. Other approaches use fragments of antibodies lacking the Fc region or antibodies that contain multiple amino acid substitutions in the Fc region. Although the anti-CD40L antibody, hu5c8, showed efficacy in human patients there is no anti-CD40L antibody on the market. Accordingly, there is a need for improved anti-CD40L antibodies for administration to humans that do not cause platelet activation or aggregation yet are stable and bind to CD40L.

### SUMMARY

The present invention provides anti-CD40L antibodies, suitable for use in humans and non-human primates, having an Fc domain that has been engineered to reduce or eliminate platelet aggregation and the concomitant risk of thromboembolism. In one aspect of the invention, the present invention provides antibodies that are humanized versions of the mouse anti-human CD40L antibody 5c8. In one embodiment, an antibody of the present invention comprises a human IgG1 consensus framework wherein the variable light chain and the variable heavy chain comprise the CDR sequences of 5c8.

One aspect of the present invention is an isolated antibody that binds to CD40L and that comprises a light chain and a heavy chain, wherein (i) the light chain comprises a light chain variable region comprising an amino acid sequence having at least 95% sequence identity with SEQ ID NO: 1; (ii) the heavy chain comprises a heavy chain variable region and an Fc region wherein a) the heavy chain variable region comprises an amino acid sequence having at least 95% sequence identity with SEQ ID NO: 2; and b) the Fc region comprises an amino acid sequence having at least 95% sequence identity with SEQ ID NO: 3 wherein the Fc region comprises one or a combination of substitutions selected from the group consisting of C11S, C14S, and P23S. Optionally the Fc region comprises a further amino acid substitution CSS.

Another aspect of the present invention is a method for treating a subject with a CD40L-associated disease or disorder comprising administering to the subject a therapeutically effective amount of an antibody according to the invention. One embodiment of the present invention is a method for treating a subject with a neurodegenerative or neuromuscular disease or disorder; an inflammatory or immune disease or disorder; or an autoimmune disease, comprising administering to the subject a therapeutically effective amount of an antibody according to the invention. Another embodiment is a method for treating a subject with a CD40L-associated disease or disorder comprising administering to the subject a therapeutically effective amount of an antibody according to the invention administered in combination with a compound that blocks the interaction between CD28 and CD86 or between CD28 and CD80.

### BRIEF DESCRIPTION OF THE FIGURES

FIGS. 1A, 1B, and 1C show the heavy chain amino acid sequences for hu5c8 (SEQ ID NO: 21) (FIG. 1A), JB5 (SEQ ID NO: 9) (FIG. 1B), and JB5-K74R (SEQ ID NO: 13) (FIG. 1C). The amino acids shown in bold type indicate amino acids that differ between the heavy chain sequences for 5c8 and the heavy chain sequences for JB5 and JB5-K74R.

FIGS. 2A-2D show the light chain amino acid sequence for JB5 (SEQ ID NO: 7) (FIG. 2A), the light chain amino acid sequence for JB5-R28K (SEQ ID NO: 11) (FIG. 2B), the Fc region amino acid sequence for hu5c8 (SEQ ID NO: 3) (FIG. 2C), and the Fc region amino acid sequence for JB5 (SEQ ID NO: 4) (FIG. 2D). The amino acids shown in bold type indicate the amino acids that differ between the light chain sequences for 5c8 and JB5-R28K and between the Fc regions for hu5c8 and JB5.

FIG. 3 is a graph showing the relative binding to human CD40L, of JB5 antibody (circles, dotted line), hu5c8 antibody (squares-solid line), and the control CTLA4-IgG1 (triangles).

FIG. 4 is a graph showing the binding of hu5c8 antibody to FCGR1A (circle, solid line) (SEQ ID NO: 22), FCGR2A (circle, dotted line) (SEQ ID NO: 23), FCR3A (SEQ ID NO: 24) and FCR3B (SEQ ID NO: 25) isoforms of the human Fc gamma receptor protein.

FIG. 5 is a graph showing that JB5 antibody to FCGR1A (SEQ ID NO: 22), FCGR2A (SEQ ID NO: 23), FCR3A (SEQ ID NO: 24), or FCR3B (SEQ ID NO: 25) isoforms of the human Fc gamma receptor protein.

FIG. 6 shows the analytical chromatography elution profile for JB5 antibody run at 30° C. from a size exclusion column.

FIG. 7 shows the analytical chromatography elution profile for hu5c8 antibody run at 30° C. from a size exclusion column.

FIG. 8 is a graph showing the binding of the platelet activation marker PAC1 antibody to untreated platelet samples (negative control), as assessed by fluorescence activated cell sorting (FACS).

FIG. 9 is a graph showing the binding, as assessed by FACS, of an anti-PAC1 antibody.

FIG. 10 is a graph showing the binding, as assessed by FACS, of an anti-PAC1 antibody to platelets after the incubation of the platelets with CD40L.

FIG. 11 is a graph showing the binding, as assessed by FACS, of an anti-PAC1 antibody to platelets after incubation of the platelets with an immune complex of CD40L and hu5c8 antibody.

FIG. 12 is a graph showing the binding, as assessed by FACS, of an anti-PAC1 antibody to platelets after incubation of the platelets with an immune complex of CD40L and JB5 antibody.

FIG. 13 is a graph showing the binding, as assessed by FACS, of an anti-PAC1 antibody to platelets after incubation of the platelets with an immune complex of CD40L and the hu5c8 F(ab′)₂.

FIG. 14 is a scatter plot graph showing FACS results from three persons' platelets after incubation of the platelets with 20 M ADP, 5 g/ml CD40L, the immune complex of CD40L and hu5c8, the immune complex of CD40L and JB5 antibody or the immune complex of CD40L with hu5c8 F(ab′)₂.

FIG. 15 provides the variable light region amino acid sequence of the anti-CD40L antibodies JB5 and hu5c8 (SEQ ID NO: 1), the variable heavy region amino acid sequence of the anti-CD40L antibodies JB5 and hu5c8 (SEQ ID NO: 2), the Fc region amino acid sequence of the anti-CD40L antibody hu5c8 (SEQ ID NO: 3), the Fc region amino acid sequence of the anti-CD40L antibody JB5 (SEQ ID NO: 4), the variable light region amino acid sequence of the anti-CD40L antibody JB5-R28K (SEQ ID NO: 5), the variable heavy region amino acid sequence of the anti-CD40L antibody JB5-K74R (SEQ ID NO: 6), and the light chain amino acid sequence of the anti-CD40L antibody JB5 (SEQ ID NO: 7).

FIG. 16 provides the light chain synthetic nucleotide sequence that encodes the anti-CD40L antibody JB5 (SEQ ID NO: 8), upper case letters represent the exons and the lower-case letters represent the intron sequences of the synthetic gene, and also provides the heavy chain amino acid sequence of the anti-CD40L antibody JB5 (SEQ ID NO: 9).

FIG. 17 provides a synthetic nucleic acid sequence that encodes the heavy chain of the anti-CD40L antibody JB5 (SEQ ID NO: 10), upper case letters represent the exons and the lower-case letters represent the intron sequences of the synthetic gene.

FIG. 18 provides the amino acid sequence of the anti-CD40L antibody JB5-R28K (SEQ ID NO: 11), a synthetic nucleic acid sequence that encodes the light chain of the anti-CD40L antibody JB5-R28K (SEQ ID NO: 12), upper case letters represent the exons and the lower case letters represent the intron sequences of the synthetic gene, and also provides the heavy chain amino acid sequence of the anti-CD40L antibody JB5-K74R (SEQ ID NO: 13).

FIG. 19 provides a synthetic nucleic acid sequence that encodes the heavy chain of the anti-CD40L antibody JB5-K74R (SEQ ID NO: 14) upper case letters represent the exons and the lower-case letters represent the intron sequences of the synthetic gene.

FIG. 20 provides the amino acid sequences of the CDRs of the heavy and light chain of the anti-CD40L antibody JB5: JB5 CDR-L1 (SEQ ID NO: 15), JB5 CDR-L2 (SEQ ID NO: 16), JB5 CDR-L3 (SEQ ID NO: 17), JB5 CDR-H1 (SEQ ID NO: 18), JB5 CDR-H2 (SEQ ID NO: 19), JB5 CDR-H3 (SEQ ID NO: 20) and the amino acid sequence of the hu5C8 heavy chain (SEQ ID NO: 21).

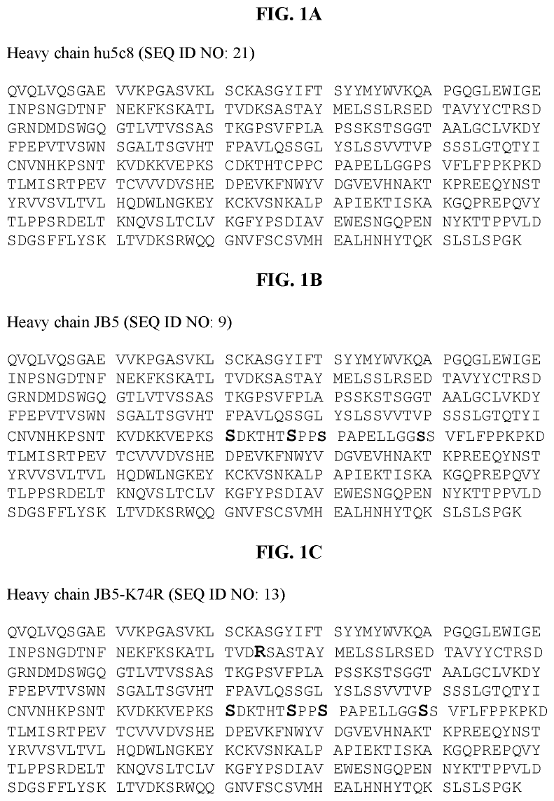

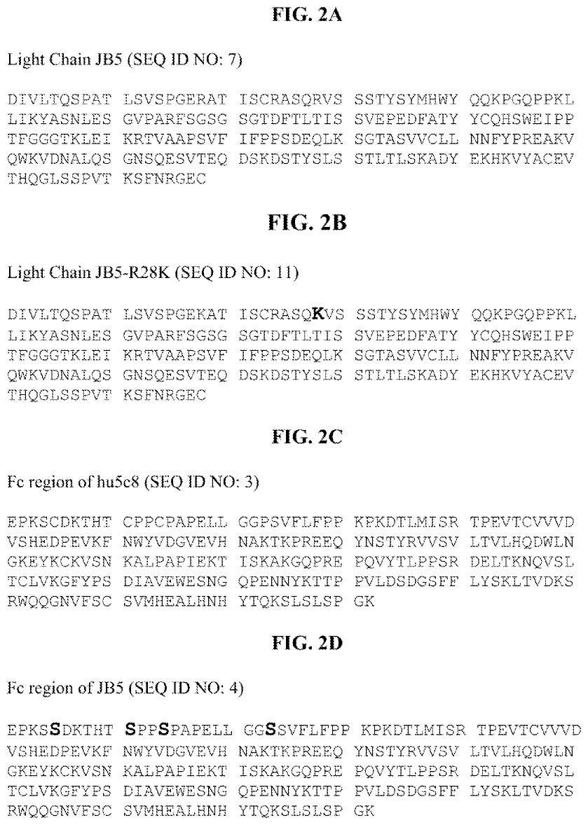

### DETAILED DESCRIPTION

### Definitions

The terms such as “comprises”, “comprised”, “comprising”, “contains”, “containing” and the like have the meaning attributed in United States patent law; these terms are inclusive or open-ended and do not exclude additional, unrecited elements or method steps. Terms such as “consisting essentially of” and “consists essentially of” have the meaning attributed to them in United States patent law; these terms allow for the inclusion of additional ingredients or steps that do not materially affect the basic and novel characteristics of the claim invention. The terms “consists of” and “consisting of” have the meaning ascribed to them in United States patent law; these terms are close ended.

The terms “treat,” “treatment” and the like, include therapeutic treatment and prophylactic treatment. Therapeutic treatment is treatment of a subject that has signs or symptoms of the disease, condition, or disorder to be treated. Prophylactic treatments refer to treatment of a subject that is predisposed to the disease, condition or disorder that does not show overt signs of the disease, condition or disorder. Thus, treatment may result in stasis of, partial or total alleviation, or reduction of signs or symptoms of illness, and specifically includes, without limitation, prolongation of survival.

“About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range.

The use of the conjunction “or” is used interchangeably with at “least one of”. For example: where a composition comprises A or B, the method must comprise at least one of A and B but may also comprise both A and B. Likewise a composition comprising “A, B, C, or D” must comprise at least one of the groups of A, B, C, and D, but may also comprise all or any combination of A, B, C, and D.

Amino acid substitutions are denoted by the convention in which the original amino acid, the position of the amino acid in the specified sequence and the replacement amino acid are identified, for example, C11S would indicate that the cysteine at position 11 of the polypeptide sequence is replaced with a serine.

“5c8” refers to the mouse anti-human antibody that binds CD40L and is produced by the hybridoma that is available from the ATCC having the accession number HB10916 and is described in U.S. Pat. No. 5,474,771. “hu5c8” refers to a humanized version of 5c8 the sequence of which is disclosed in Karpusas, et al., Structure vol. 9, pp 321-329, (2001).

Reference in the specification is made to percent identity between polypeptide or amino acid sequences. The percent identity between the two sequences is a function of the number of identical positions shared by the sequences, taking into account the number of gaps, and the length of each gap, which need to be introduced for optimal alignment of the two sequences.

Identity can be measured as “local identity” or “global identity”. Local identity refers the degree of sequence relatedness between polypeptides as determined by the match between strings of such sequences. Global identity refers to the degree of sequence relatedness of a polypeptide compared to the full-length of a reference polypeptide. Unless specified otherwise, as used herein, identity means global identity. For the purposes of this disclosure, the percentages for global identity are calculated using Needleman and Wunsch ((1970) J. Mol. Biol. 48:444-453) algorithm using a Blossum 62 scoring matrix with a gap penalty of 12, a gap extend penalty of 4, and a frameshift gap penalty of 5. There are many publicly available software programs that incorporate the Needleman and Wunsch algorithm, e.g. the GAP program in the GCG software package.

CD40L is also known as CD154, gp39, T-BAM, 5c8 antigen, or TNF related activation protein (TRAP).

### EMBODIMENTS

The present invention provides for therapeutic anti-human CD4L antibodies and methods for using the antibodies of the invention for treating patients with a CD40L-associated disease or disorder. Various exemplary embodiments of the present invention are provided, however, the invention is to be limited by the claims and not the disclosed embodiments.

In one aspect of the invention, the present invention provides antibodies that are modified versions of the anti-CD40L antibody hu5c8 that comprise a human IgG consensus framework having the variable light chain and the variable heavy chain CDR sequences of hu5c8 with an Fc domain modified to prevent platelet activation.

Table 1 provides a description of the SEQ ID NOs referenced in the application.

TABLE 1

| SEQ ID NO: | Description of Sequence |
|---|---|
| 1 | Light chain variable region amino acid sequence |
| | (hu5c8 and JBS) |
| 2 | Heavy chain variable region amino acid sequence |
| | (hu5c8 and JBS) |
| 3 | Fe region amino acid sequence (hu5c8) |
| 4 | JBS Fe region amino acid sequence |
| 5 | JBS-R28K light chain variable region amino acid |
| | sequence |
| 6 | JBS-K74R heavy chain variable region amino acid |
| | sequence |
| 7 | JBS light chain amino acid sequence |
| 8 | JBS light chain nucleic acid sequence |
| 9 | JBS heavy chain amino acid sequence |
| 10 | JBS heavy chain nucleic acid sequence |
| 11 | JBS-R28K light chain amino acid sequence |
| 12 | JBS-R28K light chain synthetic gene nucleic acid |
| | sequence |
| 13 | JBS-K74R heavy chain amino acid sequence |
| 14 | JBS-K74R heavy chain synthetic gene nucleic acid |
| | sequence |
| 15 | CDR-1 of the JBS Variable Light Chain amino acid |
| | sequence |
| 16 | CDR-2 of the JBS Variable Light Chain amino acid |
| | sequence |
| 17 | CDR-3 of the JBS Variable Light Chain amino acid |
| | sequence |
| 18 | CDR-1 of the JBS Variable Heavy Chain amino acid |
| | sequence |
| 19 | CDR-2 of the JBS Variable Heavy Chain amino acid |
| | sequence |
| 20 | CDR-3 of the JBS Variable Heavy Chain amino acid |
| | sequence |
| 21 | Hu5c8 Heavy Chain amino acid sequence |

One embodiment (embodiment A) is an isolated antibody that binds to CD40L and that comprises a light chain and a heavy chain, wherein the light chain comprises a light chain variable region comprising an amino acid sequence having at least 90%, or at least 91%, or at least 92%, or at least 93%, or at least 94%, or at least 95%, or at least 96% or at least 97%, or at least 98% or at least 99% sequence identity with SEQ ID NO: 1 and the heavy chain comprises a variable heavy chain region and an Fe region, wherein the heavy chain variable region comprises an amino acid sequence having at least 90%, or at least 91%, or at least 92%, or at least 93%, or at least 94%, or at least 95%, or at least 96%, or at least 97%, or at least 98%, or at least 99% sequence identity with SEQ ID NO: 2 and the Fe region comprises an amino acid sequence having at least at least 90%, or at least 91%, or at least 92%, or at least 93%, or at least 94%, or at least 95%, or at least 96%, or at least 97%, or at least 98%, or at least 99% sequence identity with SEQ ID NO: 3 wherein the Fe region comprises one or a combination of substitutions selected from the group consisting of C11S, C14S, and P23S.

Another embodiment (embodiment B) is an isolated antibody according to embodiment A, wherein the Fe region further comprises the amino acid substitution CSS.

In variations of the embodiments A and B the antibody comprises a light chain variable region that does not comprise any of the substitutions T33W, S26D, and Q27E.

In other variations of embodiments A and B, the light chain variable region comprises the substitution R28K.

In some variations of the embodiments of A and B, the CDRs of the heavy and light chain have the sequences listed in Table 2.

TABLE 2

| | |
|---|---|
| CDR1 light chain | ISCRASQRVSSSTVSYMH |
| | (SEQ ID NO: 15) |
| CDR2 light chain | VASNLES (SEQ ID NO: 16) |
| CDR3 light chain | QHSWEIPPT(SEQ ID NO: 17) |
| CDR1 heavy chain | SVYMY (SEQ ID NO: 18) |
| CDR2 heavy chain | EINPSNGDTNFNEKFKS |
| | (SEQ ID NO: 19) |
| CDR3 heavy chain | SDGRNDMDS(SEQ ID NO: 20) |

In yet other variation of embodiments A and B, the light chain variable region comprises the amino acid sequence ICRRASQRVSSSTYSYMH (SEQ ID NO: 15). In still other embodiments, the light chain variable region comprises the amino acid sequence ICRRASQRVSSSTYSYMH (SEQ ID NO: 15) and one or both of the amino acid sequences YASNLES (SEQ ID NO: 16) and QHSWEIPPT (SEQ ID NO: 17).

In some variations of embodiments A and B, the light chain variable region comprises the amino acid sequence of SEQ ID NO: 1. In yet other embodiments the light chain variable region consists of the amino acid of SEQ ID NO: 1. In some embodiments, the light chain consists essentially of the amino acid sequence of SEQ ID NO: 7. In other embodiments, the light chain consists of the amino acid sequence of SEQ ID NO: 7. In still other embodiments, the light chain comprises the amino acid sequence of SEQ ID NO: 11. In yet other embodiments, the light chain consists essentially of the amino acid sequence of SEQ ID NO: 11. In still other embodiments, the light chain consists of the amino acid sequence of SEQ ID NO: 11.

In other variations of the embodiments A and B, the antibody comprises a heavy chain variable region that does not comprise any of the substitutions T30H, Y33W, or S54N. In some embodiments of the antibodies of embodiments A and B, the light chain variable region does not comprise any of the substitutions T33W, S26D, and Q27E. In other variations of embodiments A and B, the light chain variable region does not comprise any of the substitutions T33W, S26D, and Q27E and the heavy chain variable region does not comprise any of the substitutions T30H, Y33W, or S54N.

In yet other variations of the embodiments A and B, the heavy chain variable region comprises the substitution K74R. In one embodiment the heavy chain variable region comprises one or any combination of the amino acid sequences SYYMY (SEQ ID NO: 18), EINPSNGDTNFNEKFKS (SEQ ID NO: 19), and SDGRNDMDS (SEQ ID NO: 20).

In another embodiment, the heavy chain variable region comprises the amino acid sequence of SEQ ID NO: 2. In yet another embodiment the heavy chain variable region consists essentially of the amino acid sequence of SEQ ID NO: 2. In still another embodiment the heavy chain variable region consists of the amino acid sequence of SEQ ID NO: 2. In some embodiments, the heavy chain variable region comprises the amino acid sequence of SEQ ID NO: 6. In yet other embodiments the heavy chain variable region consists essentially of the amino acid sequence of SEQ ID NO: 6. In still other embodiments the heavy chain variable region consists of the amino acid sequence of SEQ ID NO: 6.

One embodiment of the present invention is an isolated antibody, wherein the light chain comprises the amino acid sequence of SEQ ID NO: 1 and the heavy chain consists of the amino acid sequence of SEQ ID NO: 9.

Another embodiment of the present invention is an isolated antibody, wherein the light chain consists of the amino acid sequence of SEQ ID NO: 7 and the heavy chain consists of the amino acid sequence of SEQ ID NO: 9.

Yet another embodiment is an isolated antibody wherein the light chain variable region comprises the amino acid sequence of SEQ ID NO: 5 and the heavy chain consists of the amino acid sequence of SEQ ID NO: 9.

Still another embodiment is an isolated antibody wherein the light chain consists of the amino acid sequence of SEQ ID NO: 11 and the heavy chain consists of the amino acid sequence of SEQ ID NO: 9.

Yet another embodiment, is an isolated antibody wherein the light chain consists of the amino acid sequence of SEQ ID NO: 7 and the heavy chain consists of the amino acid sequence of SEQ ID NO: 13.

Another embodiment is an isolated antibody wherein the light chain consists of the amino acid sequence of SEQ ID NO: 11 and the heavy chain consists of the amino acid sequence of SEQ ID NO: 13.

In preferred embodiments, the antibody of the present invention is stable at 37° C. for a period of at least 12 hours.

In another aspect, the present disclosure provides methods for treating subjects having a CD40L-associated disease or disorder comprising administering to the subject a therapeutically effective amount of an antibody of the present invention. It is contemplated that an antibody of the invention, or mixtures thereof, can be administered to the subject as a monotherapy, which, as used herein, means that the antibody is the only therapeutic agent administered to the patient that is directed to the treatment of the underlying disease or disorder. Monotherapy using an antibody of the invention does not preclude the administration of other drugs, non-limiting examples of which are muscle relaxants, nonsteroidal anti-inflammatory drugs, pain medications, and antidepressants. Accordingly, in various embodiments of the invention, one or a mixture of the antibodies of the invention, is the sole therapeutic agent directed to treatment of the underlying disease or disorder.

It is also contemplated that the antibodies of the invention, or mixtures thereof, can be administered in combination with other therapeutic agents. “In combination with” includes, but is not limited to, administration of the therapeutic agents at different times, at different frequencies, simultaneously, or combined in a single dosage form.

One embodiment is a method for treating a subject with a neurodegenerative or neuromuscular disease or disorder comprising administering to the subject a therapeutically effective amount of an antibody of the present invention. Neurodegenerative or neuromuscular diseases and disorders include, but are not limited to, Alzheimer's Disease, Parkinson's Disease, Amyotrophic Lateral Sclerosis, Multifocal Motor Neuropathy, Primary Lateral Sclerosis, Spinal Muscular Atrophy, Kennedy's Disease, and Spinocerebellar Ataxia.

Another embodiment is a method for treating a subject with Amyotrophic Lateral Sclerosis comprising administering to the subject a therapeutically effective amount of an antibody of the present invention.

One embodiment of the present invention is a method for treating a subject with an inflammatory or immune disease or disorder comprising administering to the subject a therapeutically effective amount of an antibody of the present invention. Inflammatory or immune diseases and disorders include, but are not limited to, colitis, drug induced lupus nephritis, graft versus host disease, transplant rejection and atherosclerosis.

Still another embodiment is a method for treating a subject having an autoimmune disease comprising administering to the subject a therapeutically effective amount of an antibody of the present invention. Autoimmune diseases include, but are not limited to systemic lupus erythematous, type-I diabetes, myasthenia gravis, inflammatory bowel disease, immune thrombocytopenic purpura and rheumatoid arthritis.

Yet another embodiment is method of inhibiting an immune response in a subject comprising administering to the subject a therapeutically effective amount of an antibody of the present invention. In one embodiment the immune response is graft vs. host disease. In another embodiment the immune response is organ transplant rejection.

In some embodiments, an antibody of the present invention is administered as a monotherapy. In one embodiment the antibody is JBS is administered as monotherapy. In another embodiment the antibody JBS-K74R is administered as monotherapy. In yet another embodiment the antibody JBS-R28K is administered as monotherapy. In still another embodiment the antibody JBS-R28K-K74R is administered as monotherapy.

In some embodiments of the methods according to the present invention, the antibody is administered in combination with another therapeutic agent.

In some embodiments, the antibody of the present invention is administered in combination with a compound that blocks the interaction between CD28 and CD86 or between CD28 and CD80.

In some embodiments the compound that blocks the interaction between CD28 and CD86 or between CD28 and CD80 is a CTLA4-Ig fusion protein. In one embodiment the compound that blocks the interaction between CD28 and CD86 or between CD28 and CD80 is abatacept or belatacept or galiximab.

### Pharmaceutical Compositions and Methods of Administration

To treat any of the foregoing disorders, pharmaceutical compositions for use in accordance with the methods of the present disclosure may be formulated in a conventional manner using one or more physiologically acceptable carriers. Pharmaceutically acceptable carriers are determined in part by the particular composition being administered, as well as by the particular method used to administer the composition. Accordingly, there are a wide variety of suitable formulations of the compounds useful in the methods of the present disclosure (see, e.g., Remington: The Science and Practice of Pharmacy, 20th ed., Gennaro et al. Eds., Lippincott Williams and Wilkins, 2000).

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

According to the present disclosure the compounds can be administered by any suitable means, which can vary, depending on the type of disorder being treated and on the nature of the compound itself. For example, for the antibodies of the present invention, administration routes preferably include parenteral, e.g., intramuscular, intravenous, intraarterial, intraperitoneal, or subcutaneous. Preferably, the parenteral dosing is given by injection, most preferably intravenous, intramuscular or subcutaneous injection. The amount to be administered will depend on a variety of factors such as the clinical symptoms, weight of the individual, and whether other drugs are administered. It should be appreciated that determination of proper dosage forms, dosage amounts, and routes of administration is within the level of ordinary skill in the pharmaceutical and medical arts.

### Examples

The following examples illustrate the methods used to make and test the antibodies of the invention. Suitable modifications and adaptations of the described conditions and parameters normally encountered in the art of molecular biology and immunology will be apparent to one of skill in the art.

### Example 1: Antibody Production

In order to produce the antibodies of the invention, nucleic acid sequences encoding the heavy chain and the light chain of the desired antibody were designed to be suitable for expression in mammalian cells such as Chinese Hamster Ovary (CHO) cells. The nucleic acids were then artificially synthesized and ligated into the antibody expression vector BPJPuro using standard molecular biology techniques. BPJPuro is a dual gene mammalian expression vector optimized for selectable and stable expression of immunoglobulins in Chinese Hamster Ovary (CHO) cells. The vector is then transfected into CHO cells and stable transfectants selected.

### Production of JBS Antibodies

A nucleic acid (SEQ ID NO: 10) encoding a heavy chain having the amino acid sequence of SEQ ID NO: 9, and a nucleic acid (SEQ ID NO: 8) encoding a light chain having the amino acid sequence of SEQ ID NO: 7, were synthesized and ligated into the antibody expression vector BPJPuro.

The resulting expression vector encoding the heavy and light chains was transfected into the CHO line (CHO SA, Cellectis SA, Paris, France) using liposome mediated transfection.

Stable transfectants were isolated by puromycin selection and subcloned to provide clonal cell lines. Candidate cell lines were adapted to serum free suspension culture and screened for IgG production and robust growth. One of the cell lines was selected and named JBS, the cell line was cultured in a pilot scale bioreactor and the antibody JBS was purified from conditioned medium by sequential concentration, Protein A/G affinity chromatography, and size exclusion chromatography.

### Example 2: CD40L Binding Assay

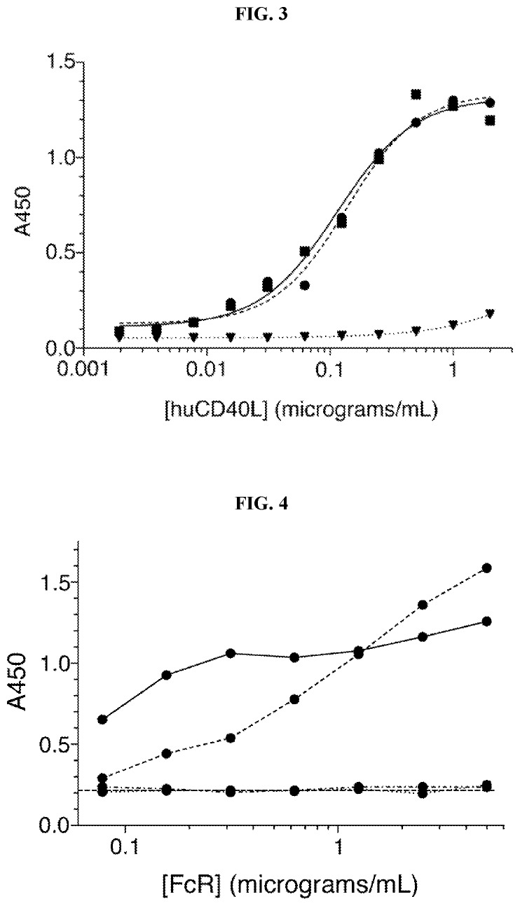

A three part sandwich ELISA assay was used to determine binding kinetics of the JBS antibody relative to the parental antibody hu5c8. All washes were performed using 3 washes of 250 μl of PBS. A 96-well polystyrene plate was coated with 100 μl/well of JBS or hu5c8 antibody (2 μg/ml) for 16 hours at 4° C. The plate was washed and then blocked with 2% bovine serum albumin/PBS for 1 hour at room temperature. The plate was washed and recombinant human CD40L protein (Santa Cruz Biotechnology, Santa Cruz, California, USA) was added to the plate titrated out by 2-fold dilution starting at 2000 ng/ml. After binding and washing, the bound CD40L protein was detected using 100 μl a biotinylated goat anti-human CD40L polyclonal antibody (200 ng/ml) and 100 μl a streptavidin-horseradish peroxidase conjugate at 100 ng/ml. Colorimetric detection was performed with the chromagen TMB (3,3′,5,5′-tetramethylbenzidine) and spectrophotometric analysis of absorption at 450 nm The resulting binding curves (FIG. 3) show that JBS (circle) has highly similar CD40L binding relative to the parental antibody hu5c8 (square). The control protein CTLA4-IgG1 (triangle), having the same Fc domain as JBS showed no significant binding. The calculated EC50 for hu5c8 and JBS is 114 and 137 nM, respectively. JB5-R28K and JB5-K74R showed binding similar to that of JBS.

### Example 3: Fe Gamma Receptor Binding Assays

hu5c8/Human Fe Gamma Receptor Binding Assay

A solid phase ELISA binding assay was performed to determine the level of binding of four human Fe gamma receptor isoforms to the parental hu5c8 antibody. 100 μI/well hu5c8 antibody (2 μg/ml in phosphate buffered saline) was added to the wells of a 96 well polystyrene plate and incubated for 16 hours at 4° C. The plate was blocked and recombinant human Fe gamma receptor (FCGR) proteins (Santa Cruz Biotechnology, Santa Cruz, California) titrated by 2-fold dilution with a starting concentration of 5 μg/ml. Four recombinant FCGR isoforms were tested separately as follows: high affinity Fc region of IgG receptor IA (FCGR1A) (CD64) (SEQ ID NO: 22), low affinity immunoglobulin gamma fc region receptor IIA (FCGR2A) (CD32) (SEQ ID NO: 23), low affinity immunoglobulin gamma fc region receptor IIIA (FCGR3A) (CD16a) (SEQ ID NO: 24), low affinity immunoglobulin gamma fc region receptor IIIB (FCGR3B) (CD16b) (SEQ ID NO: 25). After binding and washing, the FCGR was detected using an appropriate FCGR isoform specific murine monoclonal antibody (1000 ng/ml) and a horseradish peroxidase conjugate goat anti-mouse IgG detector antibody. Colorimetric detection was performed with the chromagen TMB (3,3′,5,5′-tetramethylbenzidine) and spectrophotometric analysis of absorption at 450 nm The resulting binding curves (FIG. 4) demonstrate that the parental hu5c8 antibody binds the high affinity FCGR1A (circle, solid line) receptor (SEQ ID NO: 22) and the FCGR2A receptor (SEQ ID NO: 23) (circle, dotted line) expressed on activated platelets, with high affinity. The hu5c8 antibody showed no binding to the FCGR3A receptor (SEQ ID NO: 24) or FCGR3B receptor (SEQ ID NO: 25) isoforms.

### JBS-Human Fe Gamma Receptor Binding Assay

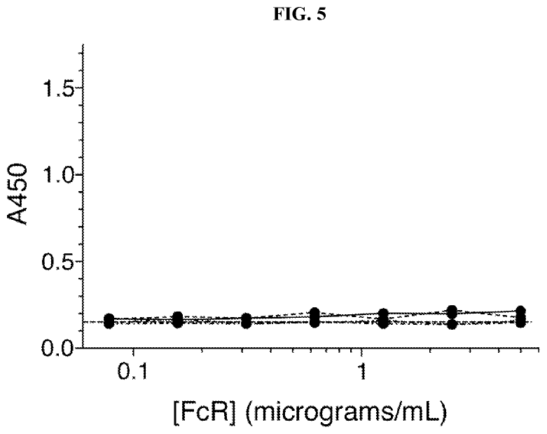

A solid phase binding assay was used to test binding of human Fe gamma receptor isoforms to the mutant JBS antibody. 100 μl/well JBS (2 μg/ml in phosphate buffered saline) was coated for 16 hours onto a 96 well polystyrene plate. The plate was blocked and recombinant human Fe gamma receptor (FCGR) proteins (Santa Cruz Biotechnology, Santa Cruz, California) titrated onto by 2-fold dilution with a starting concentration of 5 μg/ml. Four recombinant FCGR isoforms were tested separately as follows: FCGR1A (CD64) (SEQ ID NO: 22), FCGR2A (CD32) (SEQ ID NO: 23), FCGR3A (CD16a) (SEQ ID NO: 24), FCGR3B (CD16b) (SEQ ID NO: 25). After binding and washing the FCGR was detected using an appropriate FCGR isoform specific murine monoclonal antibody (1000 ng/ml) and a horseradish peroxidase conjugate goat anti-mouse IgG detector antibody. Colorimetric detection was performed with the chromagen TMB (3,3′,5,5′-tetramethylbenzidine) and spectrophotometric analysis of absorption at 450 nm The resulting binding curves (FIG. 5) demonstrate that the JBS antibody binds neither the high affinity FCGR1A receptor (SEQ ID NO: 22) nor the FCGR2A receptor (SEQ ID NO: 23), expressed on activated platelets, in this assay. Like the parental hu5c8 antibody, no binding was observed for FCGR3A receptor (SEQ ID NO: 24) or FCGR3B receptor (SEQ ID NO: 25).

### Example 4: Stability of JBS at 22° C. and at 37° C.

Because JBS lacks three of the disulfide linkages in wild-type IgG1 antibodies, JBS was tested using size exclusion chromatography to determine if the antibody was stable, i.e., existed as a tetrameric, fully intact antibody. Hu5c8, which has the three disulfide linkages was used as a control.

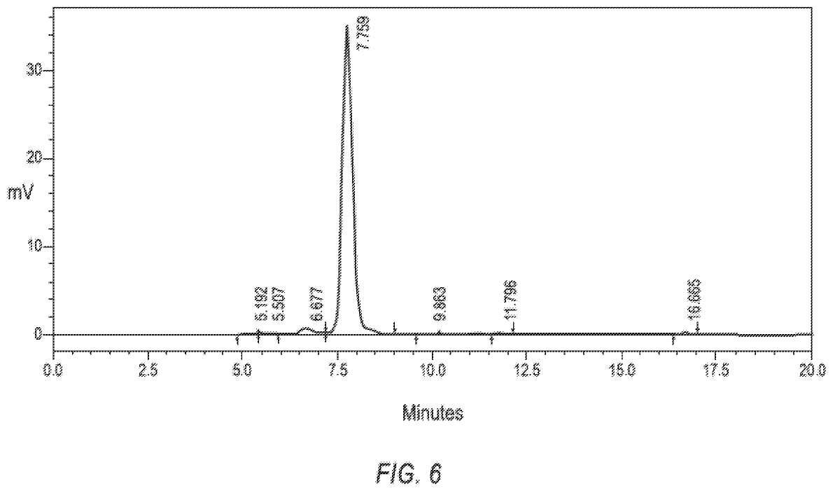

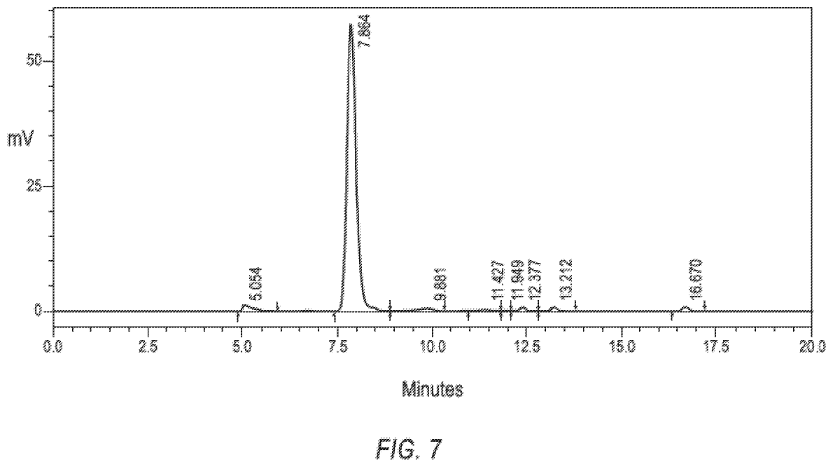

Two experiments were performed, each comparing JBS with hu5c8. In the first experiment, the antibodies were at room temperature (22° C.) before and during chromatography. To simulate in vivo conditions, in the second experiment the antibodies were incubated in human plasma at 37° C. for 30 minutes prior to chromatography at 30° C. Twenty micrograms of JBS or hu5c8 in PBS was injected into a TSK® gel G3000SW (7.8 mm×30 cm, 5 μm bead column) equipped with a pre-column filter TSK® gel Guard SW ×1, (6.0 mm×4.0 cm, 7 μm bead column) (Tosoh Bioscience, King of Prussia, PA). The mobile phase was PBS and the elution rate was 1.0 mL/minute and the absorbance was measured at 280 nm. At both 22° C. and at 30° C. JBS had an observed molecular weight of 183 kDa (FIG. 6) and hu5c8 (FIG. 7) had a MW of 164 kDa consistent with the antibody being in the tetrameric, divalent form. The observed 19 kDa difference between the hu5c8 antibody and JBS may be due to increased glycosylation of the Fc domain of JBS.

### Example 5: Elimination of Platelet Activation

In order to determine the effect of JBS on CD40L immune complex mediated platelet activation, the antibody was assayed for its ability to induce the platelet cell surface marker protein PAC-1. Whole blood was drawn from three healthy volunteers into 3.2% Na citrate tubes discarding the first 2 ml. Platelet rich plasma was prepared by centrifugation for 15 minutes at 120 g the platelet count was normalized with phosphate buffered saline to 1×10⁵ cells/ml. Immune complexes of recombinant human CD40L (Santa Cruz Biotechnology, Santa Cruz, CA, USA) and the test antibodies, hu5c8, JBS, and hu5c8 F(ab′)2 were prepared at a CD40L:Antibody molar ratio of 3:1 (0.6944 nmole CD40L:0.2315 nmole antibody) by preincubation at room temperature for 15 minutes. The immune complex mixture was diluted to a final concentration of 5 μg/ml CD40L in the normalized PBS/platelet solution and incubated at 37° C. for 30 minutes. Negative controls were untreated platelets and CD40L alone. The platelet activation positive control was prepared by the addition of ADP to a final concentration of 20 micromolar in the normalized PBS-platelet solution. After 30 minutes of incubation, anti-human PAC-1-FITC conjugated antibody was added to all samples and incubated for 15 minutes.

Samples were diluted 1:1 into 2% paraformaldehyde:PBS buffer, fixed on ice for 30 minutes, centrifuged at 100 g, for 5 minutes to pellet the cells. The cells were resuspended in PBS. Fluorescence activated cell sorting (FACS™) was performed on a Guava easyCyte™ flow cytometer (EMD Millipore, Inc., Billerica, MA, USA). Post-acquisition analysis was performed using FlowJo™ software (FlowJo, LLC, Ashland, OR, USA).

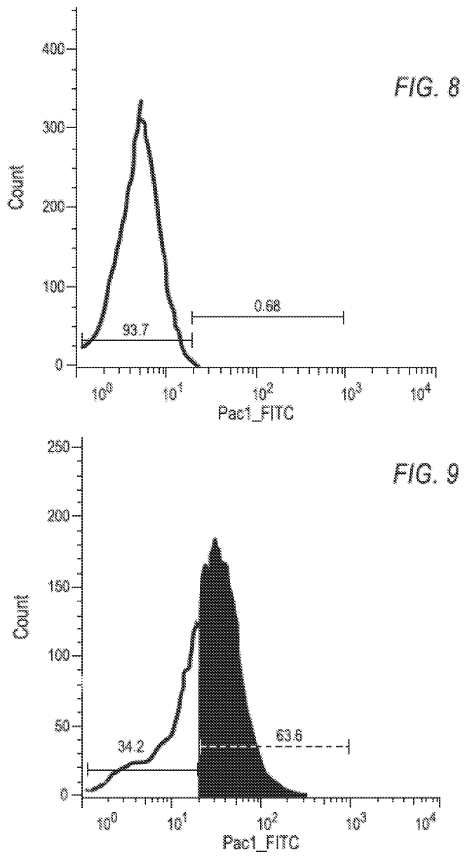

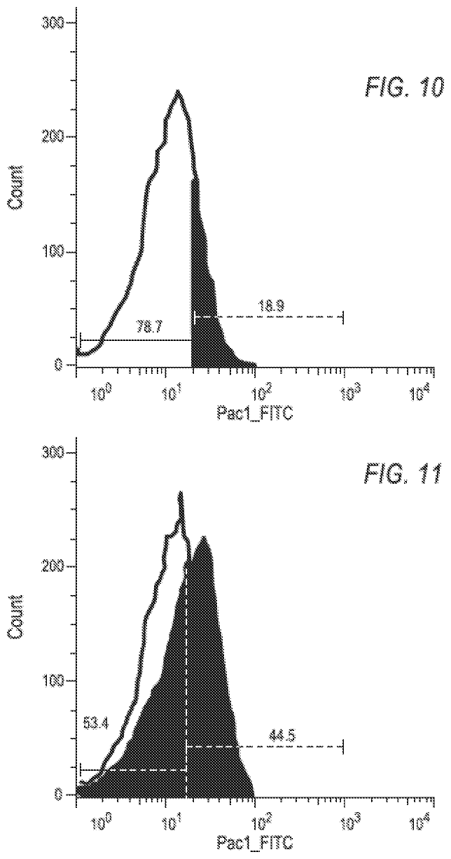

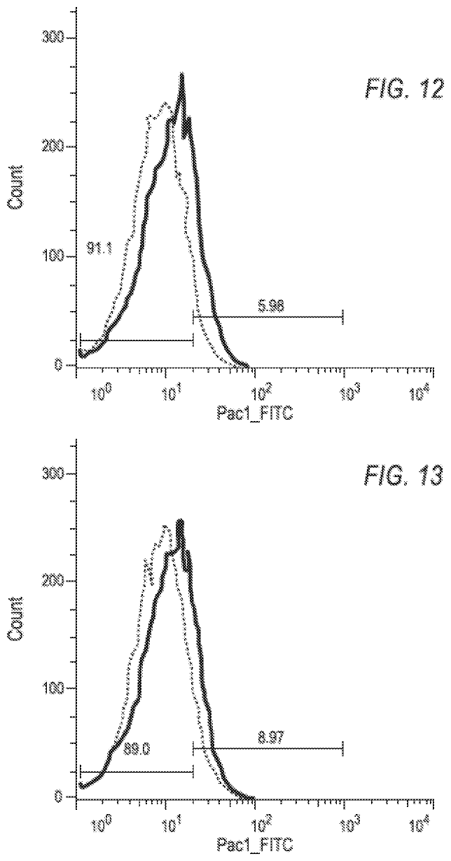

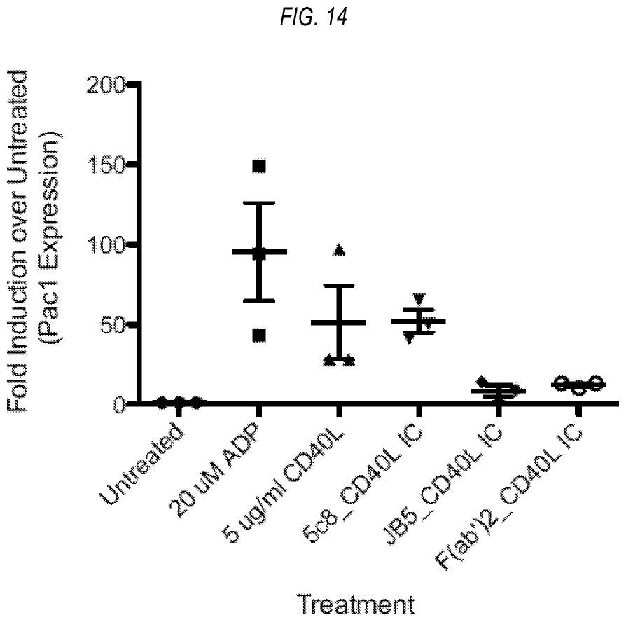

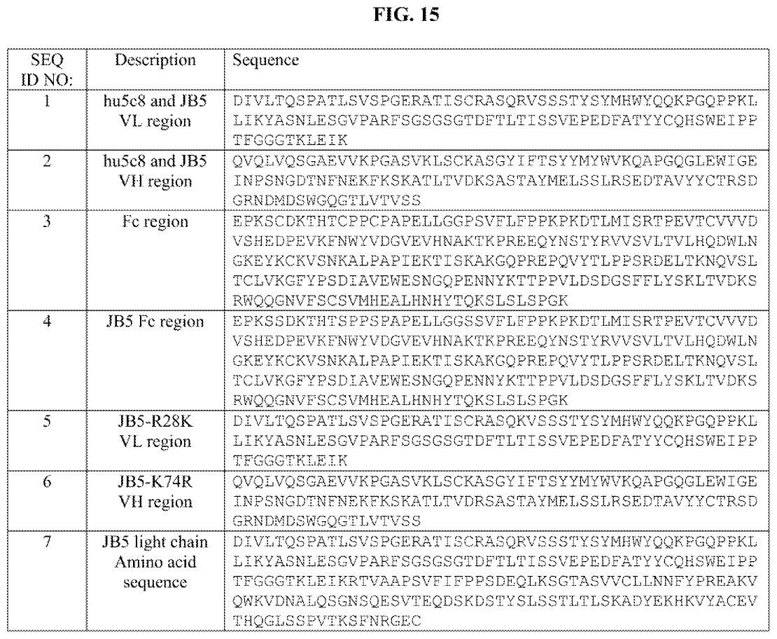

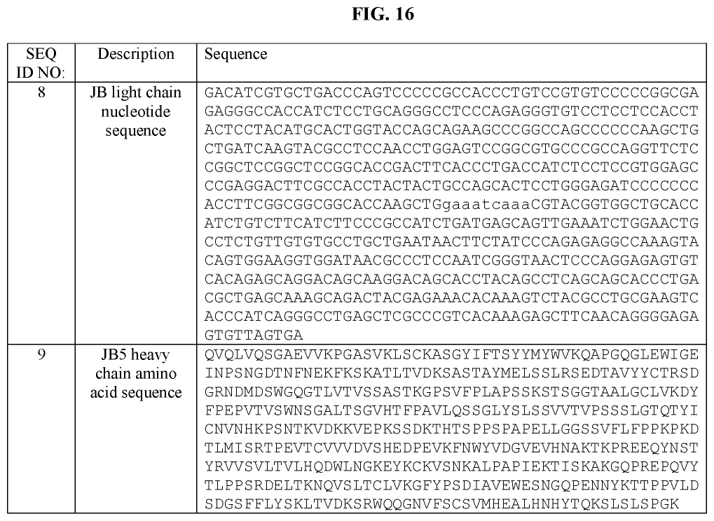

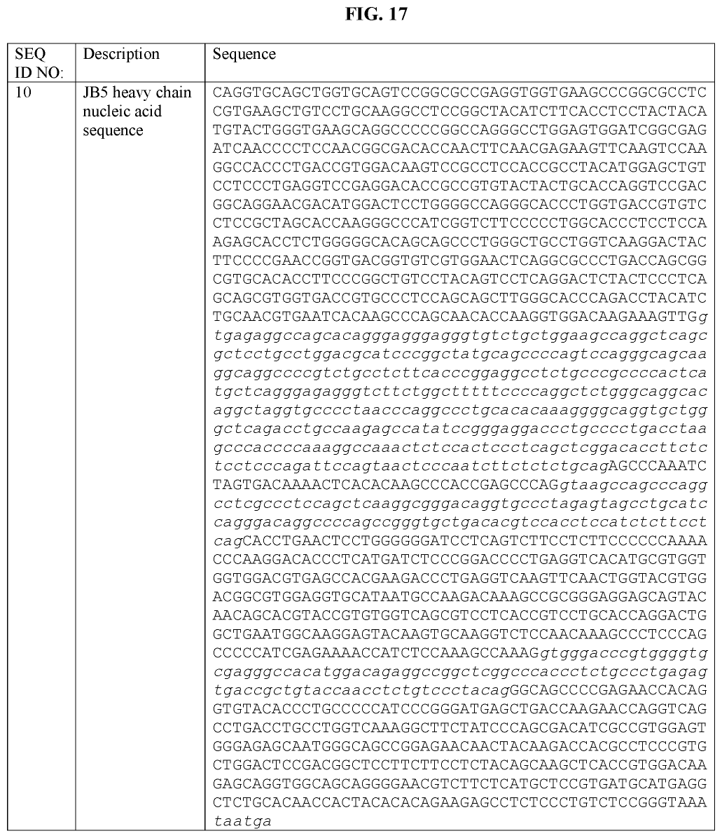

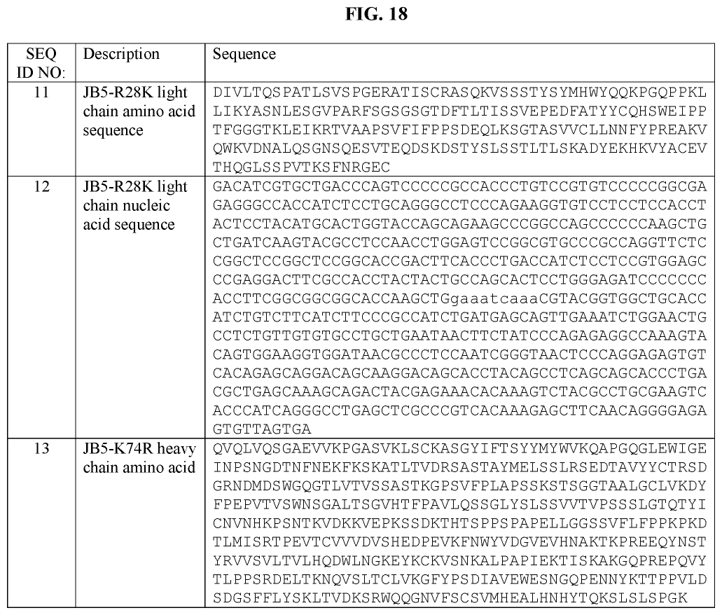

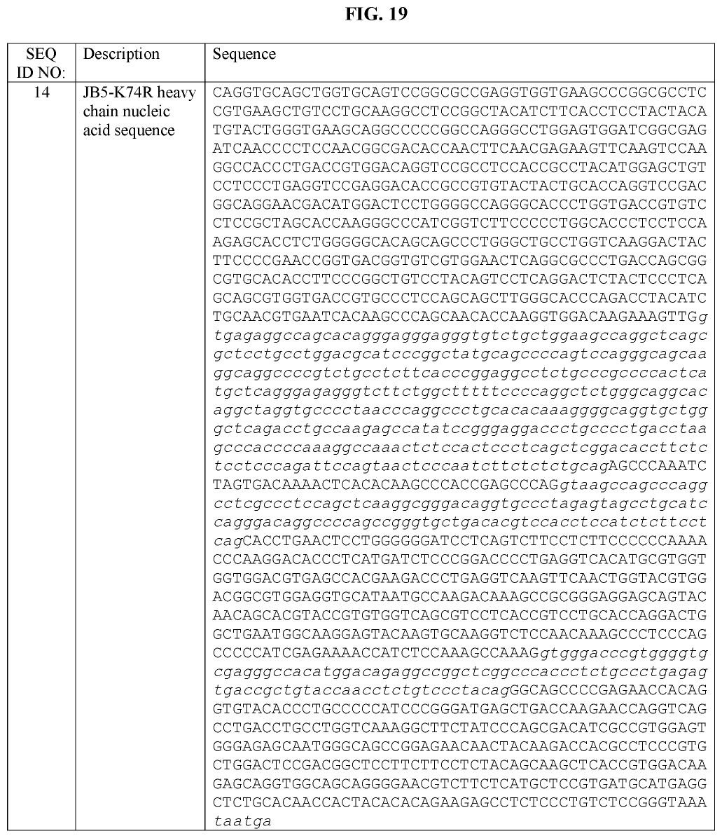

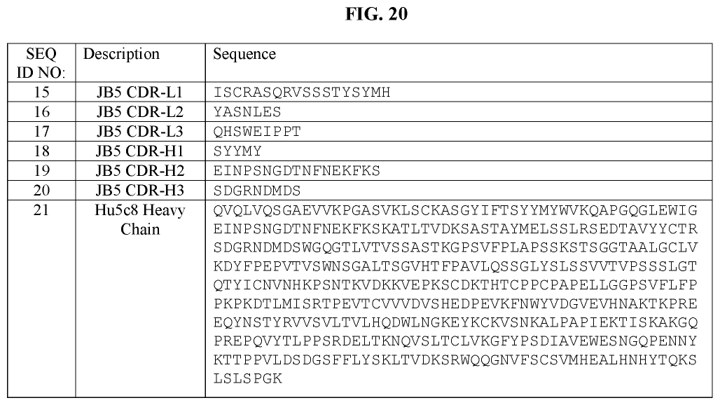

An untreated platelet control sample was used to set negative and positive PAC-1 activation gates (FIG. 8). Platelets activated with 20 micromolar ADP had a significant increase in PAC-1 cell surface expression (FIG. 9). Consistent with published observations, see e.g., Mirabet, M., et al., Molecular Immunology 45, 937-944 (2008), CD40L alone was able to activate platelets at a low level (FIG. 10). This activation was significantly increased when CD40L was present with hu5c8 antibody as an immune complex (FIG. 11). In contrast, the engineered antibody JBS complexed with CD40L demonstrated very low levels of platelet activation (FIG. 12). This reduction in the activation potential of a CD40L:JB5 immune complex is mediated by the loss of FcR interaction because the hu5c8 F(ab′)₂:CD40L immune complex (FIG. 13) also did not activate platelets relative to the hu5c8-IgG1:CD40L immune complex (FIG. 11). FIG. 14 shows the platelet activation results from three persons' platelets after incubation of the platelets with 20 μM ADP, 5 μg/ml CD40L, the immune complex of CD40L and hu5c8, the immune complex of CD40L and JBS antibody or the immune complex of CD40L with hu5c8 F(ab′)₂. The JBS immune complex showed no significant platelet activation when compared to the immune complex of CD40L with hu5c8 F(ab′)2 platelets (p<0.34 (Unpaired T test, 2 tailed; t=1.013, df=4). Further, the JBS immune complex showed significantly less platelet activation when compared with the hu5c8 immune complex (p<0.005 (Unpaired T test, 2 tailed; t=S.586, df=4).

While a number of embodiments of this disclosure are described, it is apparent that the basic examples may be altered by one skilled in the art to provide other embodiments that use or encompass methods and processes of this invention. The embodiments and examples are for illustrative purposes and are not to be interpreted as limiting the disclosure, but rather, the appended claims define the scope of this invention.

1. (canceled)

2. An isolated antibody that binds to CD40L, wherein the isolated antibody comprises a light chain, a heavy chain, and an Fc region, wherein the light chain comprises L-CDR1, L-CDR2, and L-CDR3 sequences, and the heavy chain comprises H-CDR1, H-CDR2, and H-CDR3 sequences, wherein:
the L-CDR2 comprises SEQ ID NO: 16;
the L-CDR3 comprises SEQ ID NO: 17;
the H-CDR1 comprises SEQ ID NO: 18;
the H-CDR2 comprises SEQ ID NO: 19; and
the H-CDR3 comprises SEQ ID NO: 20;
wherein the Fc region consists of the amino acid sequence of SEQ ID NO: 4.

3. The isolated antibody of claim 2, wherein the L-CDR1 comprises SEQ ID NO: 15.

4. The isolated antibody according to claim 2, wherein the antibody is stable at 37° C. for a period of at least 30 minutes.

5. A pharmaceutical composition comprising isolated antibody that binds to CD40L, wherein the isolated antibody comprises a light chain, a heavy chain, and an Fc region, wherein the light chain comprises L-CDR1, L-CDR2, and L-CDR3 sequences, and the heavy chain comprises H-CDR1, H-CDR2, and H-CDR3 sequences, wherein:
the L-CDR2 comprises SEQ ID NO: 16;
the L-CDR3 comprises SEQ ID NO: 17;
the H-CDR1 comprises SEQ ID NO: 18;
the H-CDR2 comprises SEQ ID NO: 19; and
the H-CDR3 comprises SEQ ID NO: 20;
wherein the Fc region consists of the amino acid sequence of SEQ ID NO: 4.

6. The pharmaceutical composition according to claim 5, wherein the LCDR1 comprises SEQ ID NO: 15.

7. The pharmaceutical composition according to claim 5, further comprising a physiologically acceptable carrier.

8. The pharmaceutical composition according to claim 6, further comprising a physiologically acceptable carrier.

9. A method for treating a subject with a CD40L associated disease or disorder comprising administering to the subject a therapeutically effective amount of an isolated antibody that binds to CD40L, wherein the isolated antibody comprises a light chain, a heavy chain, and an Fc region, wherein the light chain comprises L-CDR1, L-CDR2, and L-CDR3 sequences, and the heavy chain comprises H-CDR1, H-CDR2, and H-CDR3 sequences, wherein:
the L-CDR2 comprises SEQ ID NO: 16;
the L-CDR3 comprises SEQ ID NO: 17;
the H-CDR1 comprises SEQ ID NO: 18;
the H-CDR2 comprises SEQ ID NO: 19; and
the H-CDR3 comprises SEQ ID NO: 20;
wherein the Fc region consists of the amino acid sequence of SEQ ID NO: 4.

10. The method according to claim 9, wherein the LCDR1 comprises SEQ ID NO: 15.

11. The method according to claim 9, wherein the disease or disorder is selected from Amyotrophic Lateral Sclerosis, systemic lupus erythematous, type-1 diabetes, myasthenia gravis, inflammatory bowel disease, immune thrombocytopenic purpura, rheumatoid arthritis, colitis, drug induced lupus nephritis, graft versus host disease, transplant rejection and atherosclerosis, Alzheimer's Disease, Parkinson's Disease, Multifocal Motor Neuropathy, Primary Lateral Sclerosis, Spinal Muscular Atrophy, Kennedy's Disease, and Spinocerebellar Ataxia.

12. The method of claim 9, wherein the pharmaceutical composition is administered in combination with another therapeutic agent.

13. The method of claim 12, wherein the agent is abatacept, belatacept, galiximab, or a CTLA4-Ig fusion protein.

14. The method according to claim 10, wherein the disease or disorder is selected from Amyotrophic Lateral Sclerosis, systemic lupus erythematous, type-1 diabetes, myasthenia gravis, inflammatory bowel disease, immune thrombocytopenic purpura, rheumatoid arthritis, colitis, drug induced lupus nephritis, graft versus host disease, transplant rejection and atherosclerosis, Alzheimer's Disease, Parkinson's Disease, Multifocal Motor Neuropathy, Primary Lateral Sclerosis, Spinal Muscular Atrophy, Kennedy's Disease, and Spinocerebellar Ataxia.

15. The method of claim 10, wherein the pharmaceutical composition is administered in combination with another therapeutic agent.

16. The method of claim 15, wherein the agent is abatacept, belatacept, galiximab, or a CTLA4-Ig fusion protein.


SEQUENCE LISTING (25 sequences)

SEQ ID NO: 1 - protein, 111 aa, synthetic construct
  REGION 1..111: note = Synthetically Generated Sequence
DIVLTQSPATLSVSPGERATISCRASQRVSSSTYSYMHWYQQKPGQPPKLLIKYASNLESGVPARFSGSGSGTDFTLTISSVEPEDFATYYCQHSWEIPPTFGGGTKLEIK

SEQ ID NO: 2 - protein, 118 aa, synthetic construct
  REGION 1..118: note = Synthetically Generated Sequence
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSS

SEQ ID NO: 3 - protein, 232 aa, synthetic construct
  REGION 1..232: note = Synthetically Generated Sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 232 aa, synthetic construct
  REGION 1..232: note = Synthetically Generated Sequence
EPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 111 aa, synthetic construct
  REGION 1..111: note = Synthetically Generated Sequence
DIVLTQSPATLSVSPGERATISCRASQKVSSSTYSYMHWYQQKPGQPPKLLIKYASNLESGVPARFSGSGSGTDFTLTISSVEPEDFATYYCQHSWEIPPTFGGGTKLEIK

SEQ ID NO: 6 - protein, 118 aa, synthetic construct
  REGION 1..118: note = Synthetically Generated Sequence
QVQLVQSGAEVVKPGASVKLSCKASGYIFTSYYMYWVKQAPGQGLEWIGEINPSNGDTNFNEKFKSKATLTVDRSASTAYMELSSLRSEDTAVYYCTRSDGRNDMDSWGQGTLVTVSS

SEQ ID NO: 7 - protein, 218 aa, synthetic construct
  REGION 1..218: note = Synthetically Generated Sequence
DIVLTQSPATLSVSPGERATISCRASQRVSSSTYSYMHWYQQKPGQPPKLLIKYASNLESGVPARFSGSGSGTDFTLTISSVEPEDFATYYCQHSWEIPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 660 bp, synthetic construct
  misc_feature 1..660: note = Synthetically Generated Sequence
  source 1..660: mol_type = other DNA
gacatcgtgctgacccagtcccccgccaccctgtccgtgtcccccggcgagagggccaccatctcctgcagggcctcccagagggtgtcctcctccacctactcctacatgcactggtaccagcagaagcccggccagccccccaagctgctgatcaagtacgcctccaacctggagtccggcgtgcccgccaggttctccggctccggctccggcaccgacttcaccctgaccatctcctccgtggagcccgaggacttcgccacctactactgccagcactcctgggagatcccccccaccttcggcggcggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 9 - protein, 448 aa, synthetic construct
  REGION 1..448: note = Synthetically Generated Sequence
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1956 bp, synthetic construct
  misc_feature 1..1956: note = Synthetically Generated Sequence
  source 1..1956: mol_type = other DNA
caggtgcagctggtgcagtccggcgccgaggtggtgaagcccggcgcctccgtgaagctgtcctgcaaggcctccggctacatcttcacctcctactacatgtactgggtgaagcaggcccccggccagggcctggagtggatcggcgagatcaacccctccaacggcgacaccaacttcaacgagaagttcaagtccaaggccaccctgaccgtggacaagtccgcctccaccgcctacatggagctgtcctccctgaggtccgaggacaccgccgtgtactactgcaccaggtccgacggcaggaacgacatggactcctggggccagggcaccctggtgaccgtgtcctccgctagcaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatctagtgacaaaactcacacaagcccaccgagcccaggtaagccagcccaggcctcgccctccagctcaaggcgggacaggtgccctagagtagcctgcatccagggacaggccccagccgggtgctgacacgtccacctccatctcttcctcagcacctgaactcctggggggatcctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaataatga

SEQ ID NO: 11 - protein, 218 aa, synthetic construct
  REGION 1..218: note = Synthetically Generated Sequence
DIVLTQSPATLSVSPGERATISCRASQKVSSSTYSYMHWYQQKPGQPPKLLIKYASNLESGVPARFSGSGSGTDFTLTISSVEPEDFATYYCQHSWEIPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 660 bp, synthetic construct
  misc_feature 1..660: note = Synthetically Generated Sequence
  source 1..660: mol_type = other DNA
gacatcgtgctgacccagtcccccgccaccctgtccgtgtcccccggcgagagggccaccatctcctgcagggcctcccagaaggtgtcctcctccacctactcctacatgcactggtaccagcagaagcccggccagccccccaagctgctgatcaagtacgcctccaacctggagtccggcgtgcccgccaggttctccggctccggctccggcaccgacttcaccctgaccatctcctccgtggagcccgaggacttcgccacctactactgccagcactcctgggagatcccccccaccttcggcggcggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 13 - protein, 448 aa, synthetic construct
  REGION 1..448: note = Synthetically Generated Sequence
QVQLVQSGAEVVKPGASVKLSCKASGYIFTSYYMYWVKQAPGQGLEWIGEINPSNGDTNFNEKFKSKATLTVDRSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1956 bp, synthetic construct
  misc_feature 1..1956: note = Synthetically Generated Sequence
  source 1..1956: mol_type = other DNA
caggtgcagctggtgcagtccggcgccgaggtggtgaagcccggcgcctccgtgaagctgtcctgcaaggcctccggctacatcttcacctcctactacatgtactgggtgaagcaggcccccggccagggcctggagtggatcggcgagatcaacccctccaacggcgacaccaacttcaacgagaagttcaagtccaaggccaccctgaccgtggacaggtccgcctccaccgcctacatggagctgtcctccctgaggtccgaggacaccgccgtgtactactgcaccaggtccgacggcaggaacgacatggactcctggggccagggcaccctggtgaccgtgtcctccgctagcaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatctagtgacaaaactcacacaagcccaccgagcccaggtaagccagcccaggcctcgccctccagctcaaggcgggacaggtgccctagagtagcctgcatccagggacaggccccagccgggtgctgacacgtccacctccatctcttcctcagcacctgaactcctggggggatcctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaataatga

SEQ ID NO: 15 - protein, 18 aa, synthetic construct
  REGION 1..18: note = Synthetically Generated Sequence
ISCRASQRVSSSTYSYMH

SEQ ID NO: 16 - protein, 7 aa, synthetic construct
  REGION 1..7: note = Synthetically Generated Sequence
YASNLES

SEQ ID NO: 17 - protein, 9 aa, synthetic construct
  REGION 1..9: note = Synthetically Generated Sequence
QHSWEIPPT

SEQ ID NO: 18 - protein, 5 aa, synthetic construct
  REGION 1..5: note = Synthetically Generated Sequence
SYYMY

SEQ ID NO: 19 - protein, 17 aa, synthetic construct
  REGION 1..17: note = Synthetically Generated Sequence
EINPSNGDTNFNEKFKS

SEQ ID NO: 20 - protein, 9 aa, synthetic construct
  REGION 1..9: note = Synthetically Generated Sequence
SDGRNDMDS

SEQ ID NO: 21 - protein, 448 aa, synthetic construct
  REGION 1..448: note = Synthetically Generated Sequence
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 23 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 24 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 25 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI